(19) United States
(12) Patent Application Publication
(10) Pub. No.: US 2014/0166022 A1
(43) Pub. Date: Jun. 19, 2014

(54) RADIOTHERAPY PATIENT IMMOBILIZATION DEVICE AND METHOD

(71) Applicant: Norman L. Brown, Jacksonville, FL (US)

(72) Inventor: Norman L. Brown, Jacksonville, FL (US)

(21) Appl. No.: 14/159,896

(22) Filed: Jan. 21, 2014

Related U.S. Application Data
(63) Continuation-in-part of application No. 14/010,297, filed on Aug. 26, 2013.
(60) Provisional application No. 61/692,818, filed on Aug. 24, 2012.

(51) Int. Cl. A61N 5/10; A61F 5/37

(52) U.S. Cl. CPC A61N 5/10; A61F 5/3769
USPC 128/845

(57) ABSTRACT

A patient immobilization apparatus includes an inflatable shell having a patient receiving area and defines an interior volume. A fill material is arranged within the interior volume. At least one control valve located on the inflatable shell and coupled to the interior volume. The at least one control valve operates to control air introduction and evacuation into and from the interior volume. At least one is pocket located on the patient receiving area. The at least one pocket has a pocket opening and supplemental padding is arranged in the at least one pocket and is removable through the pocket opening. The inflatable shell and the fill material conform to the shape of a patient laying on the patient receiving area with the control valve open, and closing the control valve locks the inflatable shell and the fill material into their conformed shape.

### CROSS REFERENCE TO RELATED APPLICATIONS

This application is a continuation-in-part of U.S. application Ser. No. 14/010,297 filed on Aug. 26, 2013, which claims the benefit of U.S. Provisional Application Ser. No. 61/692,818 filed on Aug. 24, 2012, the contents of which applications are herein incorporated by reference in their entirety.

### FIELD OF THE INVENTION

The present invention relates generally to radiotherapy, and more specifically, to a device and method for immobilizing a patient during radiotherapy.

### BACKGROUND OF THE INVENTION

Patient positioning systems are used for accurate and reproducible positioning of a patient for radiation therapy, diagnostic imaging, surgery, and other medical procedures. During these procedures, it is important to immobilize a part or parts of the patient's body. Accurate positioning of the body part is also important in repeat treatments so that the same location of the body will be exposed to the radiation each time. Therefore, different types of devices have been made to immobilize body parts and to index the body to the treatment table to assure proper and repeatable alignment for radiation therapy. Radiation therapy has been proven effective in treating certain types of cancer if a sufficient dose of radiation is delivered directly and repeatedly to the tumor or lesion; however, complications tend to result due to damage to healthy tissue and organs surrounding the tumor. Traditional techniques used in the positioning of patients during radiation therapy treatments include use of invasive and non-invasive immobilization devices.

In invasive immobilization techniques, the immobilization device is secured to the patient in an invasive fashion, with screws, pins and rigid metal devices that are tightly attached to the patient. However, invasive techniques to immobilize a patient are risky and provide pain and discomfort for the patient.

In non-invasive immobilization techniques, a mold of the patient is typically used on which tattoos, or other markers, are placed when the patient is properly positioned upon the diagnostic table. At the time of the radiation therapy treatment, the same mold is placed upon the treatment table, and the patient is placed over the mold, after which the radiation therapy treatment begins. An example of such non-invasive apparatus that is currently being used includes the Vac-Lok™ cushion manufactured by CIVCO (Kalona, Iowa) that uses a support layer with a displaceable fill material and a protective cover. A valve extends through the protective cover into the support layer at one end with the valve exposed to the outside. The apparatus is placed on an examination table and the patient is positioned over the apparatus based on the location of the area that needs to be treated with radiotherapy. The weight of the patient forces air from the support layer and displaces the fill material, which molds around the patient to some degree. Once the patient is situated in the desired position, the valve is closed to lock the Vac-Lok™ apparatus into its molded shape. While devices like this have proved useful, further improvements are possible.

### SUMMARY OF THE INVENTION

In view of the foregoing, an object of the present invention is to provide an improved radiotherapy patient immobilization device and method of use. According to an aspect of the present invention, a patient immobilization apparatus comprises an inflatable shell defining an interior volume that contains fill material. A control valve is located on the inflatable shell to control air introduction and evacuation into and from the interior volume. When the patient lays on the shell, the fill material and shell conform to the contours of the patient's body restricting the patient's movement during the treatment. Advantageously, at least one pocket is arranged on the inflatable shell beneath a sensitive area of a patient lying on the shell. The pocket carries supplemental padding to provide additional comfort to the patient and minimizing the likelihood that the patient will move during a procedure. The pocket can also include a pocket closure to secure the supplemental padding within the pocket and prevent the supplemental padding from moving during the treatment. In one aspect of the invention, the sensitive area of the patient is the patient's thorax or pelvis. In another aspect of the invention, multiple pockets can be used to provide supplemental padding beneath the patient's arms and spine. In yet another aspect of the invention, the pocket can be arranged to provide supplemental padding beneath the patient's neck and upper-back.

According to a method aspect of the present invention, the apparatus is placed on a support surface and supplemental padding is inserted into the pocket. The control valve is opened, allowing outside air to enter the interior volume thereby inflating the inflatable shell. The patient is laid on the apparatus such that the pocket located is beneath a sensitive area of the patient. The shell and fill material conform to the contours of the patient and are locked into their conformed shape helping by closing the control valve. Additional supplemental padding may be inserted based on the patient's discomfort to minimize the likelihood that the patient will move during the procedure.

### DESCRIPTION OF THE DRAWINGS

FIG. 1 is a top view of the patient immobilizing apparatus according to an embodiment of the present invention;

FIG. 2 is a front view the patient immobilizing apparatus along the cross-section 2-2 according to an embodiment of the present invention.

FIG. 3 is a bottom view of a patient immobilizing apparatus according to an embodiment of the present invention;

FIG. 4 is a top view of the patient immobilizing apparatus according to a second embodiment of the present invention;

FIG. 5 is a top view of the patient immobilizing apparatus according to a third embodiment of the present invention;

FIG. 6 is a top view of the patient immobilizing apparatus according to a fourth embodiment of the present invention;

FIG. 7 is a top-down view of the patient immobilizing apparatus according to a fifth embodiment of the present invention.

### DETAILED DESCRIPTION OF PREFERRED EMBODIMENTS

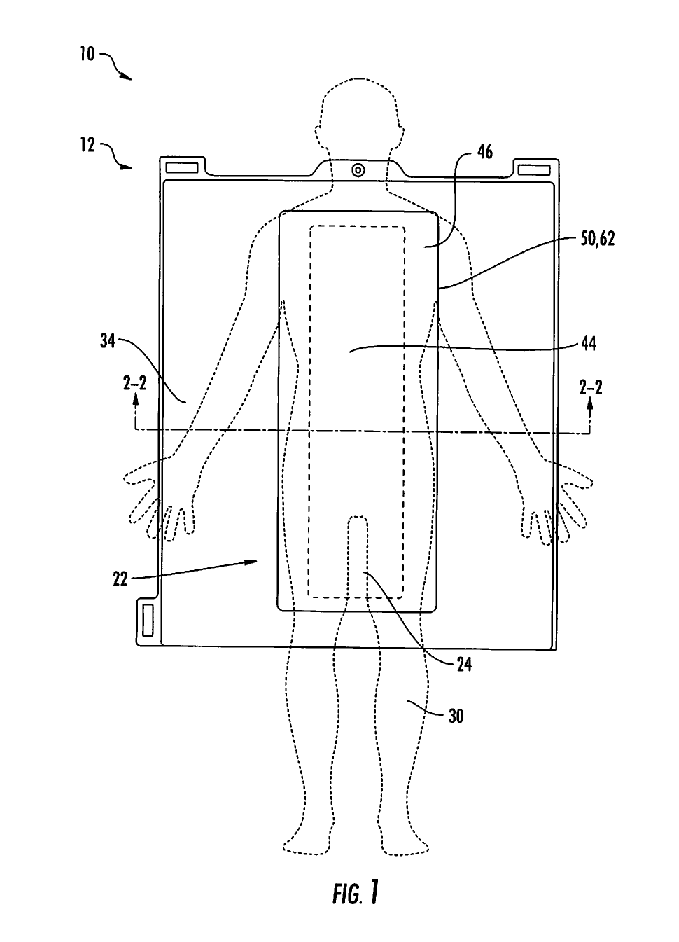

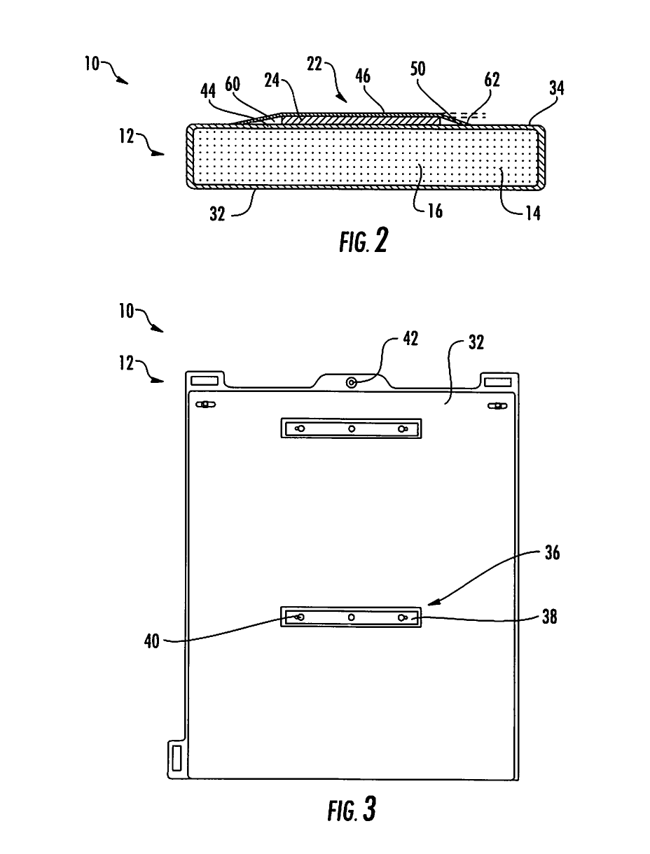

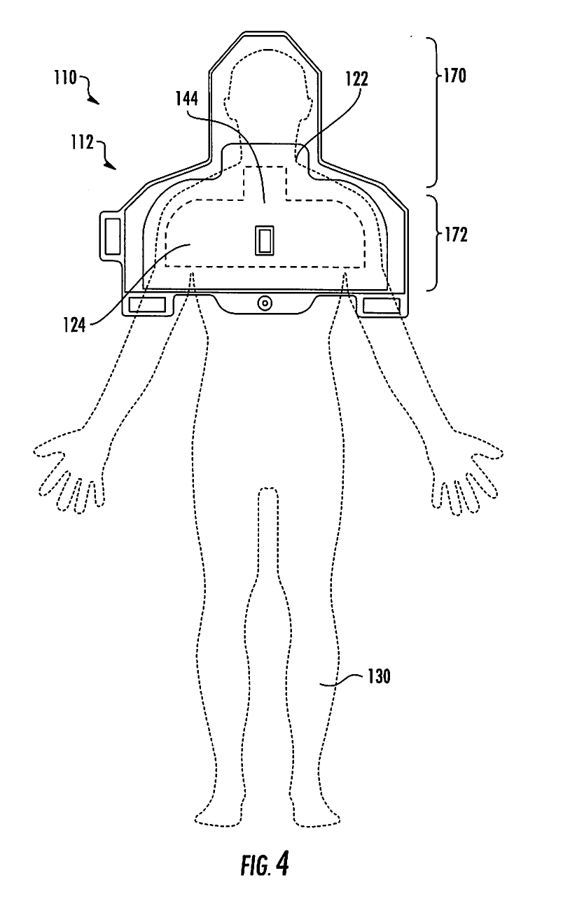

According to an embodiment of the present invention, referring to FIGS. 1-3, a patient immobilization apparatus 10 comprises a inflatable shell 12 defining an interior volume 14 containing a fill material 16, one or more control valves 20 operable to control air introduction and evacuation into and from the interior volume 14, at least one pocket 22 located on the inflatable shell 12, and supplemental padding 24 within the at least one pocket 22. In use, the apparatus 10 is placed on a support surface 26, such as an examination table or a gantry, and a patient 30 lays on the apparatus 10. With the control valve 20 open, the fill material 16 and inflatable shell 12 conform to the contours of the patient 30 laying thereon, helping immobilize the patient 30. Closing the control valve 20 vacuum locks the inflatable shell 12 and fill material 16 into their conformed shape. The pocket 22 ensures the supplemental padding 24 remains located under a sensitive point of the patient 30, minimizing patient discomfort and decreasing the likelihood of patient movement during a procedure.

The inflatable shell 12 has a lower surface 32 and an upper surface 34 defining the interior volume 14 therebetween. For referential purposes, the lower surface 32 refers to the surface of the inflatable shell 12 proximate the support surface 30 when the apparatus 10 is in use, while the upper surface 34 refers to the surface of the inflatable shell 12 proximate the patient 30 when the patient is lying on the apparatus 10. The upper and lower surfaces 32, 34 can be formed of separate pieces of material connected along perimeters thereof. Alternatively, the inflatable shell may be a single contiguous sheet of material. Preferably, the inflatable shell 12 is made from of an air-tight flexible material, such as polyurethane or the like to allow the inflatable shell 12 to form to the contours of the patient 30 lying thereon. In the embodiment depicted, the inflatable shell 12 is rectangular in shape to support with the pocket 22 positioned to support the patient's thorax or pelvis. However, it will be appreciated that alternate inflatable shell shapes and pocket configurations are contemplated based on the area of the patient to be supported/immobilized.

Fill material 16 is distributed throughout the interior volume 14. When the patient 30 lays on the inflatable shell 12 with the control valve 20 open, the fill material 14 conforms to the contours of the patient 30 in cooperation with the inflatable shell 12. The fill material 16 advantageously includes a plurality of polystyrene beads; however, other free-flowing materials may be used. The fill material can provide some cushioning effect to help minimize patient discomfort.

The control valve 20 extends through the lower surface 32 and communicates with the interior volume 14. When the control valve 20 is open, outside air enters the interior volume 14 through the control valve 20 until interior volume 14 air pressure is in equilibrium with the outside air. Closing the control valve 20 vacuum locks the inflatable shell 12 and the fill material 16 into their conformed shape helping to immobilize the patient. The lower surface 32 also carries one or more battens 36. The battens 36 each comprise a strip of rigid material 38 having a plurality of holes 40 allowing for the apparatus 10 to be secured to the support surface 26 and preventing the apparatus 10 from being moved relative to the support surface 26. A grommet 42 allows for easy storage of the apparatus 10.

The upper surface 34 comprises a patient receiving area 44 that the patient 30 contacts when laying on the inflatable shell 12. Advantageously, the pocket 22 is arranged on the patient receiving area 44 to secure the supplemental padding 24 beneath a sensitive area of the patient 30 (in the depicted embodiment, the patient's thorax or pelvis). The supplemental padding 24 provides additional cushioning to a likely sensitive area and reduces the likelihood the patient 30 will move during the procedure. The supplemental padding 24 may be made of foam or other suitable cushioning material.

The pocket 22 includes a pocket panel 46 and a pocket closure 50. The pocket panel 46 is connected to the upper surface 34 to define a pocket volume 60 therebetween with an opening 62 on an edge thereof. Alternatively, the pocket 22 may be a separate unit that is releasably connected to the upper surface 34. The pocket volume 60 houses the supplemental padding 24 and is accessible through the opening 62 for the insertion and removal of the supplemental padding 24. As one in the art will appreciate, this configuration allows for the supplemental padding 24 to be removed, or for additional supplemental padding 24 to be inserted, based on the needs of the patient 30. Additionally, the supplemental padding 24 can easily be replaced as it deteriorates over time due to radiation exposure and/or other factors.

The pocket closure 56 is releasably connected to the pocket panel 46 proximate the opening 62 and functions to close the opening 62 and secure the supplemental padding 24 within the pocket volume 14. The pocket closure 56 preferably includes complimentary hook and loop fastener strips connected to the pocket closure 56 and the upper surface 34, respectively. Other fastening devices could be used, as well, such as adhesive straps, button fasteners, or clasp fasteners. Alternately, the pocket closure 56 may consist of a flap connected to the pocket closure 56 proximate the opening 62, or be omitted altogether.

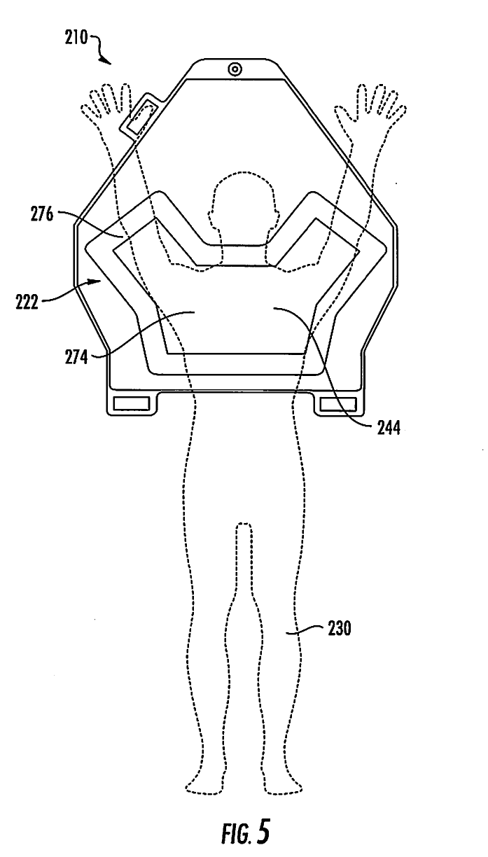

The foregoing embodiment is provided for illustrative and exemplary purposes; the present invention is not necessarily limited thereto. Some alternative embodiments of the invention will be described in connection with FIGS. 4-7. Where not specifically described, similarly numbered components of the alternative embodiments are analogous to those described above.

Referring to FIG. 4, the patient immobilizing apparatus 110 is configured to provide support to the head and neck of the patient 130. The inflatable shell 112 includes a head section 170 and an upper-back section 172 with the patient receiving area 144 located thereon to receive the head and upper-back of the patient 130, respectively. The head and upper-back sections 170,172 are generally trapezoidal in shape. Advantageously, the pocket 122 is arranged on the patient receiving area 144 proximate the upper-back section 172 to secure supplemental padding 124 under the upper back and neck of the patient 130 for additional cushioning.

Referring to FIG. 5, the patient immobilizing apparatus 210 is configured to provide support across the upper back (or front) and shoulders of the patient 230. The pocket 222 includes a central pocket area 274, and opposing pocket extensions 276 extending at an angle from opposite ends of the central pocket area 274. Advantageously, pocket 222 is arranged on the patient receiving area 244 such that the central pocket area 274 secures supplemental padding 224 beneath the patient's 230 upper-back and the opposing pocket extensions 276 secure supplemental padding 224 beneath the patient's 230 arms (when in the raised position).

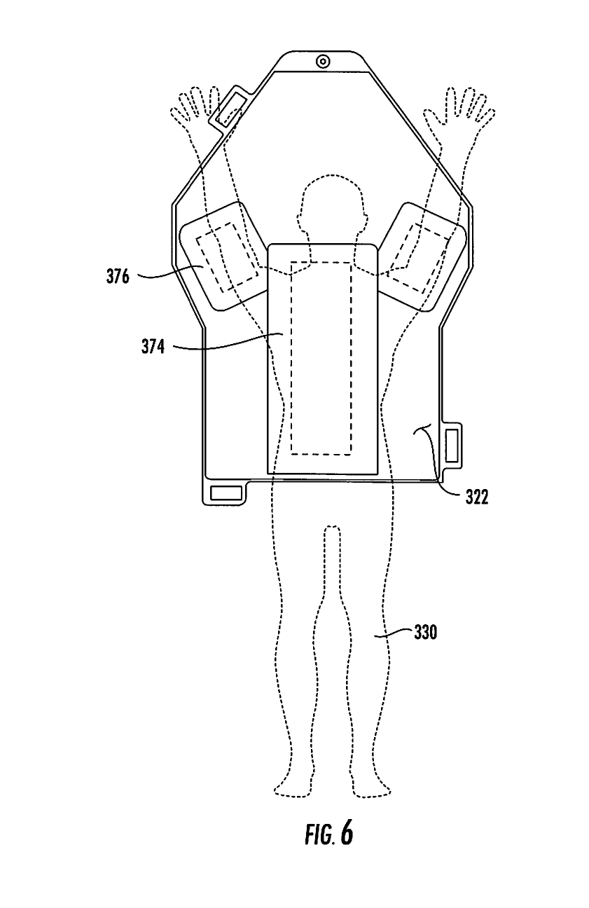

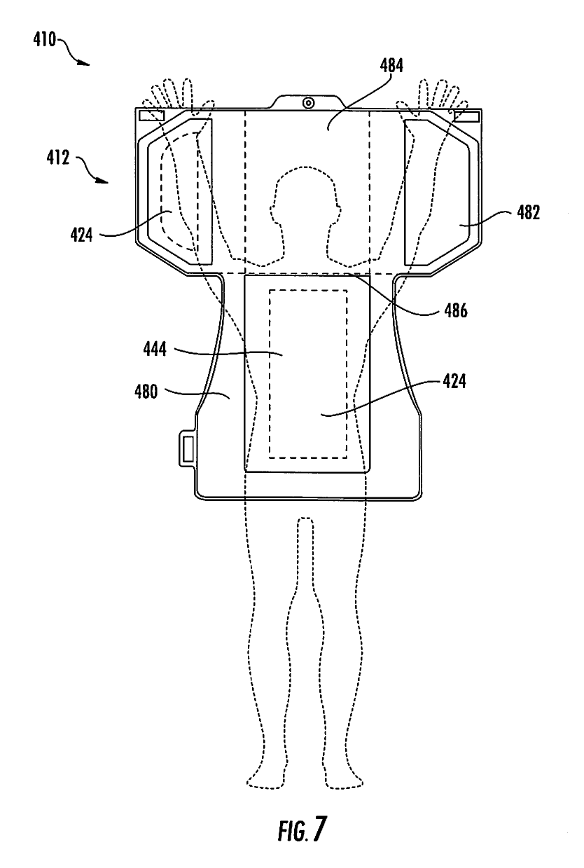

Alternatively, referring to FIG. 6, the central pocket area 374 and opposing pocket extensions 376 can be separate pockets allowing for a more customizable level of cushioning. Additionally, the central pocket area 374 may have an extended length allowing the pocket 322 to also support a patient's 330 spine.

Referring to FIG. 7, the patient immobilizing apparatus 410 is configured to provide support across the upper back (or front) and shoulders of the patient 430. Advantageously, the inflatable shell 412 has a central section 480 with two arm support sections 482 connected at opposing ends thereto. The pocket 422 is arranged on the patient receiving area 444 proximate the central section 480 to secure supplemental padding 424 beneath the patient's 430 spine. Two additional pockets 482 are each arranged on the patient receiving area 444 proximate the two arm support sections 482 to secure supplemental padding 424 beneath the patient's arms (when in the raised position).

The interior volume 414 is separated into a plurality of compartments 484 by air-permeable baffles 486. The air-permeable baffles 86 allow for the movement of air, while restricting the movement of the fill material 416, between the plurality of compartments 484. It will be appreciated that such a configuration ensures that the fill material 416 remains distributed throughout the interior volume 14 and eliminates the need to redistribute the fill materials 416 prior to use. Another benefit is to ensure that the fill material 16 conforms to all contours of the patient's 430 body by preventing the complete displacement of fill material 416 from any one of the plurality of compartments 84 when the patient initially lays down. The air-permeable baffles 486 are preferably made of a flexible mesh. Other multi-compartment configurations are possible depending on the fill material 416 used, such as a series of bladders connected by valves.

In use, the apparatus 10 is placed on a support surface and secured in place via the one or more battens 36. Supplemental padding 24 is inserted into the pocket 22 and the pocket closure 50 is closed securing the supplemental padding 24 within the pocket 22. The at least one control valve 20 is opened (if closed), allowing outside air to enter the interior volume 14 until the air in the interior volume is in equilibrium with outside air thereby inflating the inflatable shell 12. The patient 30 is laid on the patient receiving area 44 such that the pocket 22 is beneath a sensitive area of the patient 30 and the shell 12 and fill material 16 conform to the contours of the patient 30. The at least one control valve 20 is then closed locking the shell 12 and fill material 16 into the conformed shape and helping immobilize the patient by supporting them comfortably in the desired position. Additional supplemental padding may be inserted or the supplemental padding 24 may be removed based on the patient's discomfort to minimize the likelihood that the patient will move during the procedure.

In general, the foregoing description is provided for exemplary and illustrative purposes; the present invention is not necessarily limited thereto. Rather, those skilled in the art will appreciate that additional modifications, as well as adaptations for particular circumstances, will fall within the scope of the invention as herein shown and described and of the claims appended hereto.

What is claimed is:

1. A patient immobilization apparatus comprising:
an inflatable shell having a patient receiving area and defining an interior volume;
a fill material arranged within the interior volume;
at least one control valve located on the inflatable shell and coupled to the interior volume operable to control air introduction and evacuation into and from the interior volume; and
at least one pocket located on the patient receiving area and having a pocket opening; and
supplemental padding arranged in the at least one pocket and removable through the pocket opening;
wherein the inflatable shell and the fill material conform to the shape of a patient laying on the patient receiving area with the control valve open, and closing the control valve locks the inflatable shell and the fill material into their conformed shape.

2. The patient immobilization apparatus of claim 1, wherein the at least one pocket comprises:
a panel coupled to the inflatable shell defining a pocket volume therebetween in which the supplemental padding is located.

3. The patient immobilization apparatus of claim 2, wherein the pocket opening is defined between an edge of the pocket panel and the shell.

4. The patient immobilization apparatus of claim 2, wherein the at least one pocket further includes a pocket closure coupled to the pocket for releasably securing the pocket opening.

5. The patient immobilization apparatus of claim 4, wherein the pocket closure includes a flap coupled to the panel proximate the opening.

6. The patient immobilization apparatus of claim 4, wherein the pocket closure includes a hook and loop fastener.

7. The patient immobilization apparatus of claim 1, wherein the pocket is permanently affixed to the inflatable shell.

8. The patient immobilization apparatus of claim 1, wherein the interior volume comprises a plurality of compartments, each compartment coupled to each other compartment, wherein the fill material is contained within the plurality of compartments.

9. The patient immobilization apparatus of claim 8, further comprising a plurality of air-permeable baffles arranged within the interior volume and defining the plurality of compartments.

10. The patient immobilization apparatus of claim 1, wherein the at least one pocket is arranged on the patient receiving area so as to lie beneath a sensitive area of the patient when placed thereon.

11. The patient immobilization apparatus of claim 10, wherein the at least one pocket is arranged on the patient receiving area so as to lie beneath a thorax or pelvis of the patient when placed thereon.

12. The patient immobilization apparatus of claim 10, wherein the at least one pocket further includes a central pocket area and a plurality of pocket extensions coupled to the central pocket area and extending at an angle therefrom.

13. The patient immobilization apparatus of claim 12, wherein the at least one pocket arranged on the patient receiving area so the central pocket area lies beneath an upper back of the patient when placed thereon and the plurality of pocket extensions lie beneath arms of the patient when placed thereon.

14. The patient immobilization apparatus of claim 10, further comprising;
a pair of opposing arm support pockets, each of the arm support pockets containing the supplemental padding therein;
wherein the at least one pocket is arranged on the patient receiving area so as to lie beneath a spine of the patient when placed thereon;
wherein the arm support pockets are arranged on the patient receiving area so as to lie beneath arms of the patient when placed thereon.

15. The patient immobilization apparatus of claim 14, wherein the inflatable shell further includes a central area and two arm support members, the two arm support members adjacent to the central area on opposite sides thereof, wherein the at least one pocket is arranged on the patient receiving area proximate the central area, wherein each arm support pocket is arranged on the patient receiving area proximate each arm support member.

16. The patient immobilization apparatus of claim 10, the at least one pocket is arranged on the patient receiving area so as to lie beneath a neck and an upper-back of the patient when placed thereon.

17. A method of using a patient immobilization apparatus to immobilize a patient, comprising:
inserting supplemental padding into at least one pocket located on a patient receiving area of an inflatable shell;
placing a patient onto the patient receiving area such that a sensitive area of the patient is supported by the at least one pocket;
conforming the inflatable shell and fill material located therein to the patient's contours as air exits an interior volume of the inflatable shell via a control valve located on the inflatable shell;
closing the control valve to vacuum lock the inflatable shell and the fill material into their conformed shape.

18. The method of claim 17, wherein inserting the supplemental padding into the at least one pocket comprises inserting the supplemental padding into a pocket volume defined between a panel connected to the inflatable shell through an opening along an edge of the pocket volume.

19. The method of claim 18, further comprising securing the supplemental padding in the pocket volume via closing a pocket closure proximate the opening.

20. The method of claim 17, further comprising securing the inflatable shell to a support table via at least one batten located on the inflatable shell.
